(19) United States
(12) Patent Application Publication
(10) Pub. No.: US 2026/0115124 A1
(43) Pub. Date: Apr. 30, 2026

(54) FRESHENING COMPOSITION

(71) Applicant: The Procter & Gamble Company, Cincinnati, OH (US)

(72) Inventors: Julie Ann O'NEIL, Dillsboro, IN (US); Steven Anthony HORENZIAK, Cincinnati, OH (US)

(21) Appl. No.: 19/367,959

(22) Filed: Oct. 24, 2025

Related U.S. Application Data
(60) Provisional application No. 63/711,913, filed on Oct. 25, 2024.

(51) Int. Cl. A61K 8/73; A61K 8/34; A61Q 13/00

(52) U.S. Cl. CPC A61K 8/738; A61K 8/345; A61Q 13/00

(57) ABSTRACT

A composition includes water; a eutectic solvent; and unmodified beta cyclodextrin. A method of making a composition includes combining a hydrogen bond acceptor, a hydrogen bond donor, and unmodified beta cyclodextrin to form a eutectic solvent, wherein the hydrogen bond acceptor comprises a monosaccharide, a disaccharide, or a sugar alcohol; and adding the eutectic solvent to water and a perfume, forming the composition.

### TECHNICAL FIELD

The present disclosure is related to compositions having water, unmodified beta cyclodextrin, and a eutectic composition.

### BACKGROUND

Sprayable water-based compositions may be used to provide perfume to the air and/or surfaces to provide a desirable scent. Conventional sprayable water-based perfume compositions may use modified cyclodextrins, such as hydroxypropyl-beta-cyclodextrin (HPBCD), hydroxypropyl-gamma-cyclodextrin, hydroxypropyl-alpha-cyclodextrin, or methyl-beta-cyclodextrin. However, modified cyclodextrins may be undesirable due to environmental regulations. Unmodified cyclodextrins, such as alpha-cyclodextrin, beta-cyclodextrin, or gamma-cyclodextrin, may be used instead of modified cyclodextrins. However, the use of unmodified cyclodextrins poses a challenge because unmodified cyclodextrins can be difficult to incorporate into water-based formulations due to low solubility in low temperature (e.g. 20-25° C.), moderate pH (2-10) systems. Therefore, a need exists for water-based composition that can incorporate unmodified cyclodextrins at room temperature and at moderate pH. The present disclosure addresses this need by providing water-based compositions including a eutectic solvent that solubilizes unmodified cyclodextrins in aqueous compositions.

### SUMMARY

According to a first example of the present disclosure a composition includes water; perfume; a eutectic solvent; and unmodified beta cyclodextrin.

In accordance with another example of the present disclosure, a method of making a composition includes combining a hydrogen bond acceptor, a hydrogen bond donor, and unmodified beta cyclodextrin to form a eutectic solvent. The hydrogen bond acceptor comprises a monosaccharide, a disaccharide, or a sugar alcohol. The method further includes adding the eutectic solvent to water and a perfume, forming the composition.

### DETAILED DESCRIPTION

The present disclosure is related to compositions and methods of making compositions that include water, a eutectic solvent, and perfume. The eutectic solvent may solubilize the perfume in the water-based composition, leading to a reduced need for other perfume solubilizing aids, such as solvents including ethanol, surfactants, or both. In examples, the compositions including eutectic solvents of the present disclosure may have no ethanol. Additionally or alternatively, the compositions including eutectic solvents of the present disclosure may have no anionic surfactant. Additionally or alternatively, the compositions including eutectic solvents of the present disclosure may have reduced amounts of nonionic surfactant as compared to water-based compositions having solubilized perfume that do not use eutectic solvents.

The composition may be a liquid composition. The composition may be used as an aerosolized spray or as a liquid spray. The composition may be an air freshening and/or fabric freshening composition, a hard surface composition, a dish composition, an insect repellent composition, a disinfecting composition, a hair care composition, a body care composition, an antiperspirant or deodorant or the like. The composition may be an air and/or fabric freshening composition.

### Water

The composition of the present disclosure may include water. The water may be distilled, deionized, tap, or further purified forms of water. The composition may include from 50 to 99 wt %, from 60 to 99 wt %, from 70 to 99 wt %, from 80 to 99 wt %, from 85 to 99 wt %, from 90 to 99 wt %, from 95 to 99 wt %, from 97 to 99 wt %, from 50 to 97 wt %, from 60 to 97 wt %, from 70 to 97 wt %, from 80 to 97 wt %, from 85 to 97 wt %, from 90 to 97 wt %, from 95 to 97 wt %, from 50 to 95 wt %, from 60 to 95 wt %, from 70 to 95 wt %, from 80 to 95 wt %, from 85 to 95 wt %, from 90 to 95 wt %, from 50 to 90 wt %, from 60 to 90 wt %, from 70 to 90 wt %, from 80 to 90 wt %, from 85 to 90 wt %, from 90 to 90 wt %, from 95 to 90 wt %, or any values within the foregoing ranges or any ranges created thereby, of water.

### Eutectic Solvent

The composition may include a eutectic solvent. Eutectic solvents may include two or more materials that form a mixture with a melting point lower than each of the individual substances. Preferred eutectic mixtures may be liquids at temperatures less than 30° C., less than 25° C., or preferably less than 20° C., or less than 10° C., or less than 5° C.

The eutectic solvent may include at least one hydrogen bond acceptor (HBA). Hydrogen bond acceptors are atoms or groups of atoms within a molecule that can form a hydrogen bond with a hydrogen atom that is covalently bonded to another atom. In a hydrogen bond, a hydrogen atom from the donor acts as a bridge between the donor atom, typically oxygen or nitrogen, and the acceptor atom. The acceptor atom must have a lone pair of electrons available to form a bond with the hydrogen atom. The hydrogen bond acceptor may include a monosaccharide, a disaccharide, or a sugar alcohol. The monosaccharide, disaccharide, or sugar alcohol may include fructose, glucose, sucrose, xylose, sorbitol, mannose, galactose, glycerol, xylitol, or combinations thereof.

The composition may further include a hydrogen bond donor (HBD). Hydrogen bond donors are atoms or groups of atoms within a molecule that have a covalently bonded hydrogen atom capable of forming a hydrogen bond with an acceptor atom. In a hydrogen bond, the hydrogen atom acts as the donor by sharing its partially positive charge with the acceptor atom, typically oxygen, nitrogen, or fluorine. The hydrogen bond donor must have a hydrogen atom directly bonded to a highly electronegative atom, creating a partial positive charge on the hydrogen atom.

Common examples of hydrogen bond donors include hydroxyl groups (—OH), amino groups (—NH2), and carboxylic acid groups (—COOH). These groups have a hydrogen atom directly bonded to an oxygen or nitrogen atom, which can form a hydrogen bond with an electron-rich atom or group. The presence of a hydrogen bond donor in a molecule allows for the formation of intermolecular or intramolecular hydrogen bonds.

The hydrogen bond donor may include an organic hydroxy acid. The organic hydroxy acid may include lactic acid, malic acid, tartaric acid, mandelic acid, citric acid, salicylic acid, glycolic acid, 5 octanoyl salicylic acid, levulinic acid, hydroxyoctanoic acid, hydroxycaprylic acid, lanolin fatty acids, and combinations thereof.

The mechanism of eutectic solvent formation involves the establishment of strong hydrogen bonding interactions between the HBD and HBA components. The HBD donates a hydrogen atom to the HBA, forming a hydrogen bond. This hydrogen bond network may be responsible for the unique properties of eutectic solvents, including their low melting points, high thermal stability, low volatility, and excellent solvation capabilities.

The formation of hydrogen bonds in eutectic solvents allows them to solvate a wide range of compounds, including polar and non-polar substances. Without intending to be bound by theory, the solvation ability of eutectic solvents arises from the combination of the HBD's ability to interact with polar molecules through hydrogen bonding and the HBA's ability to interact with non-polar molecules through weak electrostatic interactions.

The hydrogen bond donor to hydrogen bond acceptor may be present at a molar ratio of from about 10:1 to about 1:1, from about 10:1 to about 2:1, from about 10:1 to about 4:1, from about 8:1 to about 2:1, from about 8:1 to about 4:1, from about 6:1 to about 2:1, from about 6:1 to about 4:1, about 5:1, or any values within the foregoing ranges or any ranges created thereby. The hydrogen bond donor to hydrogen bond acceptor may be present at a molar ratio of from about 1:10 to about 1:1, from about 1:10 to about 1:2, from about 1:10 to about 1:4, from about 1:8 to about 1:2, from about 1:8 to about 1:4, from about 1:6 to about 1:2, from about 1:6 to about 1:4, about 1:5, or any values within the foregoing ranges or any ranges created thereby.

The composition may include from 0.1 to 5 wt %, from 0.1 to 4 wt %, from 0.1 to 3 wt %, from 0.1 to 2.5 wt %, from 0.1 to 2 wt %, from 0.1 to 1.5 wt %, from 0.3 to 5 wt %, from 0.3 to 4 wt %, from 0.3 to 3 wt %, from 0.3 to 2.5 wt %, from 0.3 to 2 wt %, from 0.3 to 1.5 wt %, from 0.5 to 5 wt %, from 0.5 to 4 wt %, from 0.5 to 3 wt %, from 0.5 to 2.5 wt %, from 0.5 to 2 wt %, from 0.5 to 1.5 wt %, about 1 wt %, about 2 wt %, or any values within the foregoing ranges or any ranges created thereby, of active eutectic solvent.

### Perfume

The composition may include a perfume mixture comprising at least one perfume raw material (PRM). Various PRMs may be used. The composition may include a perfume mixture comprising one or more of the following perfume raw materials. As used herein, a “perfume raw material” refers to one or more of the following ingredients: fragrant essential oils; aroma compounds; pro-perfumes; materials supplied with the fragrant essential oils, aroma compounds, and/or pro-perfumes, including stabilizers, diluents, processing agents, and contaminants; and any material that commonly accompanies fragrant essential oils, aroma compounds, and/or pro-perfumes.

The composition may include from 0.1 to 5 wt %, from 0.1 to 4 wt %, from 0.1 to 3 wt %, from 0.1 to 2 wt %, from 0.2 to 5 wt %, from 0.2 to 4 wt %, from 0.2 to 3 wt %, from 0.2 to 2 wt %, from 0.5 to 5 wt %, from 0.5 to 4 wt %, from 0.5 to 3 wt %, from 0.5 to 2 wt %, from 1 to 5 wt %, from 1 to 4 wt %, from 1 to 3 wt %, from 1 to 2 wt %, from 1.5 to 5 wt %, from 1.5 to 4 wt %, from 1.5 to 3 wt %, or from 1.5 to 2 wt %, or any values within the foregoing ranges or any ranges created thereby, of perfume.

The PRM may include one or more ketones. The PRM comprising ketone can comprise any PRMs which contain one or more ketone moieties and which can impart a desirable scent. The PRM may comprise ketone comprising a PRM selected from the group consisting of buccoxime; iso jasmone; methyl beta naphthyl ketone; musk indanone; tonalid/musk plus; alpha-damascone, beta-damascone, delta-damascone, iso-damascone, damascenone, damarose, methyl-dihydrojasmonate, menthone, carvone, camphor, fenchone, alpha-ionone, beta-ionone, dihydro-beta-ionone, gamma-methyl so-called ionone, fleuramone, dihydrojasmone, cis-jasmone, iso-e-super, methyl-cedrenyl-ketone or methyl-cedrylone, acetophenone, methyl-acetophenone, para-methoxy-acetophenone, methyl-beta-naphtyl-ketone, benzyl-acetone, benzophenone, para-hydroxy-phenyl-butanone, celery ketone or livescone, 6-isopropyldecahydro-2-naphtone, dimethyl-octenone, freskomenthe, 4-(1-ethoxyvinyl)-3,3,5,5-tetramethyl-cyclohexanone, methyl-heptenone, 2-(2-(4-methyl-3-cyclohexen-1-yl) propyl)-cyclopentanone, 1-(p-menthen-6 (2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4 (5h)-indanone, 4-damascol, dulcinyl or cassione, gelsone, hexalon, isocyclemone e, methyl cyclocitrone, methyl-lavender-ketone, orivon, para-tertiary-butyl-cyclohexanone, verdone, delphone, muscone, neobutenone, plicatone, veloutone, 2,4,4,7-tetramethyl-oct-6-en-3-one, tetrameran, hedione, floralozone, gamma undecalactone, ethylene brassylate, pentadecanolide, methyl nonyl ketone, cyclopentadecanone, cyclic ethylene dodecanedioate, 3,4,5,6-tetrahydropseudoionone, 8-hexadecenolide, dihydrojasmone, 5-cyclohexadecenone, and a combination thereof.

The PRM comprising ketone may comprise a PRM selected from the group consisting of alpha-damascone, delta-damascone, iso-damascone, carvone, gamma-methyl-ionone, beta-ionone, iso-e-super, 2,4,4,7-tetramethyl-oct-6-en-3-one, benzyl acetone, beta-damascone, damascenone, methyl dihydrojasmonate, methyl cedrylone, hedione, floralozone, and a combination thereof.

The composition may include a mixture of aldehydes that contribute to scent character and neutralize malodors in vapor and/or liquid phase via chemical reactions. Aldehydes that are partially reactive or volatile may be considered a reactive aldehyde as used herein. Reactive aldehydes may react with amine-based odors, following the path of Schiff-base formation. Reactive aldehydes may also react with sulfur-based odors, forming thiol acetals, hemi thiolacetals, and thiol esters in vapor and/or liquid phase, in air or on surfaces. It may be desirable for these vapor and/or liquid phase reactive aldehydes to have virtually no negative impact on the desired perfume character, color or stability of a product.

The composition may include a mixture of aldehydes that are partially volatile which may be considered a volatile aldehyde as used herein. The volatile aldehydes may also have a certain boiling point (B.P.) and octanol/water partition coefficient (P). The boiling point referred to herein is measured under normal standard pressure of 760 mmHg. The boiling points of many volatile aldehydes, at standard 760 mm Hg are given in, for example, “Perfume and Flavor Chemicals (Aroma Chemicals),” written and published by Steffen Arctander, 1969.

The value of the log of the Octanol/Water Partition Coefficient (log P) is computed for each PRM in the perfume mixture being tested. The C log P of an individual PRM is calculated using the Consensus log P Computational Model, version 14.02 (Linux) available from Advanced Chemistry Development Inc. (ACD/Labs) (Toronto, Canada) to provide the unitless log P value. The ACD/Labs' Consensus log P Computational Model is part of the ACD/Labs model suite. The C log P values, which are the most reliable and widely used estimates for this physicochemical property, are preferably used instead of the experimental log P values in the selection of volatile aldehydes for the malodor control composition.

The C log P values may be defined by four groups and the volatile aldehydes may be selected from one or more of these groups. The first group comprises volatile aldehydes that have a B.P. of about 250° C. or less and C log P of about 3 or less. The second group comprises volatile aldehydes that have a B.P. of 250° C. or less and C log P of 3.0 or more. The third group comprises volatile aldehydes that have a B.P. of 250° C. or more and C log P of 3.0 or less. The fourth group comprises volatile aldehydes that have a B.P. of 250° C. or more and C log P of 3.0 or more. The composition may comprise any combination of volatile aldehydes from one or more of the C log P groups.

The composition may comprise, by total weight of the composition, from about 0% to about 30% of volatile aldehydes from group 1, alternatively about 25%; and/or about 0% to about 10% of volatile aldehydes from group 2, alternatively about 10%; and/or from about 10% to about 30% of volatile aldehydes from group 3, alternatively about 30%; and/or from about 35% to about 60% of volatile aldehydes from group 4, alternatively about 35%.

Exemplary reactive and/or volatile aldehydes which may be used in a composition include, but are not limited to, Adoxal (2,6,10-Trimethyl-9-undecenal), Bourgeonal (4-t-butylbenzenepropionaldehyde), Lilestralis 33 (2-methyl-4-t-butylphenyl) propanal), Cinnamic aldehyde, cinnamaldehyde (phenyl propenal, 3-phenyl-2-propenal), Citral, Geranial, Neral (dimethyloctadienal, 3,7-dimethyl-2,6-octadien-1-al), Cyclal C (2,4-dimethyl-3-cyclohexen-1-carbaldehyde), Florhydral (3-(3-Isopropyl-phenyl)-butyraldehyde), Citronellal (3,7-dimethyl 6-octenal), Cymal, cyclamen aldehyde, Cyclosal, Lime aldehyde (Alpha-methyl-p-isopropyl phenyl propyl aldehyde), Methyl Nonyl Acetaldehyde, aldehyde C12 MNA (2-methyl-1-undecanal), Hydroxycitronellal, citronellal hydrate (7-hydroxy-3,7-dimethyl octan-1-al), Helional (3-(1,3-Benzodioxol-5-yl)-2-methylpropanal; 2-Methyl-3-(3,4-methylenedioxyphenyl) propanal), Intreleven aldehyde (undec-10-en-1-al), Ligustral, Trivertal (2,4-dimethyl-3-cyclohexene-1-carboxaldehyde), Jasmorange, satinaldehyde (2-methyl-3-tolylproionaldehyde, 4-dimethylbenzenepropanal), Lyral (4-(4-hydroxy-4-methyl pentyl)-3-cyclohexene-1-carboxaldehyde), Melonal (2,6-Dimethyl-5-Heptenal), Methoxy Melonal (6-methoxy-2,6-dimethylheptanal), methoxycinnamaldehyde (trans-4-methoxycinnamaldehyde), Myrac aldehyde isohexenyl cyclohexenyl-carboxaldehyde, trifernal ((3-methyl-4-phenyl propanal, 3-phenyl lysmeral, benzenepropanal (4-tert-butyl-alpha-methyl-butanal), lilial, P.T. Bucinal, hydrocinnamaldehyde), Dupical, tricyclodecylidenebutanal (4-Tricyclo5210-2,6decylidene-8butanal), Melafleur (1,2,3,4,5,6,7,8-octahydro-8,8-dimethyl-2-naphthaldehyde), Methyl Octyl Acetaldehyde, aldehyde C-11 MOA (2-mehtyl deca-1-al), Onicidal (2,6,10-trimethyl-5,9-undecadien-1-al), Citronellyl oxyacetaldehyde, Muguet aldehyde 50 (3,7-dimethyl-6-octenyl)oxyacetaldehyde), phenylacetaldehyde, Mefranal (3-methyl-5-phenyl pentanal), Triplal, Vertocitral dimethyl tetrahydrobenzene aldehyde (2,4-dimethyl-3-cyclohexene-1-carboxaldehyde), 2-phenylproprionaldehyde, Hydrotropaldehyde, Canthoxal, anisylpropanal 4-methoxy-alpha-methyl benzenepropanal (2-anisylidene propanal), Cylcemone A (1,2,3,4,5,6,7,8-octahydro-8,8-dimethyl-2-naphthaldehyde), and Precylcemone B (1-cyclohexene-1-carboxaldehyde).

Still other exemplary aldehydes include, but are not limited to, acetaldehyde (ethanal), pentanal, valeraldehyde, amylaldehyde, Scentenal (octahydro-5-methoxy-4,7-Methano-1H-indene-2-carboxaldehyde), propionaldehyde (propanal), Cyclocitral, beta-cyclocitral, (2,6,6-trimethyl-1-cyclohexene-1-acetaldehyde), Iso Cyclocitral (2,4,6-trimethyl-3-cyclohexene-1-carboxaldehyde), isobutyraldehyde, butyraldehyde, isovaleraldehyde (3-methyl butyraldehyde), methylbutyraldehyde (2-methyl butyraldehyde, 2-methyl butanal), Dihydrocitronellal (3,7-dimethyl octan-1-al), 2-Ethylbutyraldehyde, 3-Methyl-2-butenal, 2-Methylpentanal, 2-Methyl Valeraldehyde, Hexenal (2-hexenal, trans-2-hexenal), Heptanal, Octanal, Nonanal, Decanal, Lauric aldehyde, Tridecanal, 2-Dodecanal, Methylthiobutanal, Glutaraldehyde, Pentanedial, Glutaric aldehyde, Heptenal, cis or trans-Heptenal, Undecenal (2-, 10-), 2,4-octadienal, Nonenal (2-, 6-), Decenal (2-, 4-), 2,4-hexadienal, 2,4-Decadienal, 2,6-Nonadienal, Octenal, 2,6-dimethyl 5-heptenal, 2-isopropyl-5-methyl-2-hexenal, Trifernal, beta methyl Benzenepropanal, 2,6,6-Trimethyl-1-cyclohexene-1-acetaldehyde, phenyl Butenal (2-phenyl 2-butenal), 2.Methyl-3 (p-isopropylphenyl)-propionaldehyde, 3-(p-isopropylphenyl)-propionaldehyde, p-Tolylacetaldehyde (4-methylphenylacetaldehyde), Anisaldehyde (p-methoxybenzene aldehyde), Benzaldehyde, Vernaldehyde (1-Methyl-4-(4-methylpentyl)-3-cyclohexenecarbaldehyde), Heliotropin (piperonal) 3,4-Methylene dioxy benzaldehyde, alpha-Amylcinnamic aldehyde, 2-pentyl-3-phenylpropenoic aldehyde, Vanillin (4-methoxy 3-hydroxy benzaldehyde), Ethyl vanillin (3-ethoxy 4-hydroxybenzaldehyde), Hexyl Cinnamic aldehyde, Jasmonal H (alpha-n-hexyl-cinnamaldehyde), Floralozone, (para-ethyl-alpha,alpha-dimethyl Hydrocinnamaldehyde), Acalea (p-methyl-alpha-pentylcinnamaldehyde), methylcinnamaldehyde, alpha-Methylcinnamaldehyde (2-methyl 3-pheny propenal), alpha-hexylcinnamaldehyde (2-hexyl 3-phenyl propenal), Salicylaldehyde (2-hydroxy benzaldehyde), 4-ethyl benzaldehyde, Cuminaldehyde (4-isopropyl benzaldehyde), Ethoxybenzaldehyde, 2,4-dimethylbenzaldehyde, Veratraldehyde (3,4-dimethoxybenzaldehyde), Syringaldehyde (3,5-dimethoxy 4-hydroxybenzaldehyde), Catechaldehyde (3,4-dihydroxybenzaldehyde), Safranal (2,6,6-trimethyl-1,3-diene methanal), Myrtenal (pin-2-ene-1-carbaldehyde), Perillaldehyde L-4 (1-methylethenyl)-1-cyclohexene-1-carboxaldehyde), 2,4-Dimethyl-3-cyclohexene carboxaldehyde, 2-Methyl-2-pentenal, 2-methylpentenal, pyruvaldehyde, formyl Tricyclodecan, Mandarin aldehyde, Cyclemax, Pino acetaldehyde, Corps Iris, Maceal, and Corps 4322.

### Low Molecular Weight Polyols.

The composition may comprise polyols. Low molecular weight polyols with relatively high boiling points, as compared to water, such as ethylene glycol, diethylene glycol, triethylene glycol, propylene glycol, dipropylene glycol, and/or glycerine may be utilized as a malodor counteractant for improving odor neutralization of the composition. Some polyols, e.g., dipropylene glycol, are also useful to facilitate the solubilization of some perfume ingredients in the composition.

The polyol may be glycerine, ethylene glycol, propylene glycol, dipropylene glycol, polyethylene glycol, or mixtures thereof. The polyol used may be diethylene glycol.

A low molecular weight polyol may be added to the composition at a level of from about 0.01% to about 5%, by weight of the composition, alternatively from about 0.05% to about 1%, alternatively from about 0.1% to about 0.5%, by weight of the composition. Compositions with higher concentrations of low molecular weight polyols may make fabrics susceptible to soiling and/or leave unacceptable visible stains on fabrics as the solution evaporates off of the fabric.

### Glycol Ethers

The composition may include one or more glycol ethers. Glycol ethers may be useful to facilitate the solubilization of some perfume ingredients in the composition. Examples of suitable glycol ethers may include propylene glycol methyl ether, propylene glycol phenyl ether, propylene glycol methyl ether acetate, dipropylene glycol methyl ether acetate, propylene glycol n-butyl ether, dipropylene glycol n-butyl ether, dipropylene glycol n-propyl ether, ethylene glycole phenyl ether, diethylene glycol n-butyl ether, dipropylene glycol n-butyl ether, diethylene glycol mono butyl ether, dipropylene glycol methyl ether, tripropylene glycol methyl ether, tripropylene glycol n-butyl ether, other glycol ethers, or mixtures thereof.

### Cyclodextrin

The composition may include at least one cyclodextrin. As used herein, the term “cyclodextrin” includes any of the known cyclodextrins such as unsubstituted cyclodextrins containing from six to twelve glucose units, especially alpha-cyclodextrin (ACD), beta-cyclodextrin (BCD), and gamma-cyclodextrin (available as Cavamax® from Wacker). Cyclodextrin may also include the derivatives of the aforementioned “unmodified” cyclodextrins, such as hydroxypropyl-beta-cyclodextrin (HPBCD), hydroxypropyl-gamma-cyclodextrin, hydroxypropyl-alpha-cyclodextrin, or methyl-beta-cyclodextrin (available as Cavasol® from Wacker) and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many molecules can fit into the cavity.

Cyclodextrin molecules are described in U.S. Pat. Nos. 5,714,137, and 5,942,217. Cyclodextrin may be present at from about 0.01% to about 5%, from about 0.01% to about 4%, from about 0.01% to about 3%, from about 0.01% to about 2%, from about 0.01% to about 1%, from about 0.01% to about 0.5%, from about 0.01% to about 0.3%, from about 0.01% to about 0.25%, from about 0.05% to about 5%, from about 0.05% to about 4%, from about 0.05% to about 3%, from about 0.05% to about 2%, from about 0.05% to about 1%, from about 0.05% to about 0.5%, from about 0.05% to about 0.3%, from about 0.05% to about 0.25%, from about 0.1% to about 5%, from about 0.1% to about 4%, from about 0.1% to about 3%, from about 0.1% to about 2%, from about 0.1% to about 1%, from about 0.1% to about 0.5%, from about 0.1% to about 0.3%, from about 0.1% to about 0.25%, or any values within the foregoing ranges or any ranges created thereby, by weight of the composition. Compositions with higher concentrations of cyclodextrins can make fabrics susceptible to soiling and/or leave unacceptable visible stains on fabrics as the solution evaporates off of the fabric. The latter is especially a problem on thin, colored, synthetic fabrics. In order to avoid or minimize the occurrence of fabric staining, the fabric may be treated at a level of less than about 5 mg of cyclodextrin per mg of fabric, alternatively less than about 2 mg of cyclodextrin per mg of fabric.

The cyclodextrin used in the present invention may comprise a cyclodextrin with a water solubility of less than 25 g in 100 ml of water, or less than 20 g in 100 ml of water, or less than 10 g in 100 ml of water at 25° C., which may be described in this disclosure as “low solubility cyclodextrin.” The cyclodextrin may be unmodified beta cyclodextrin.

### Surfactant

The composition may contain a solubilizing aid to solubilize any excess hydrophobic organic materials, particularly any PRMs, and also optional ingredients (e.g., insect repelling agent, antioxidant, etc.) which can be added to the composition, that are not readily soluble in the composition, to form a clear solution. A suitable solubilizing aid is a surfactant, such as a no-foaming or low-foaming surfactant. Suitable surfactants are anionic surfactants, nonionic surfactants, cationic surfactants, amphoteric surfactants, zwitterionic surfactants, and mixtures thereof.

The composition may contain nonionic surfactants, cationic surfactants, and mixtures thereof. The composition may contain surfactant derivatives of hydrogenated castor oil. Suitable ethoxylated hydrogenated castor oils that may be used in the present composition include Emulan™, Cremophor, Dehymuls or Eumulgin available from BASF.

The composition may include water-soluble nonionic surfactants. The water-soluble nonionic surfactants may include the primary aliphatic alcohol ethoxylates, secondary aliphatic alcohol ethoxylates, alkylphenol ethoxylates and ethylene-oxide-propylene oxide condensates on primary alkanols, such a PLURAFAC™ surfactants (BASF) and condensates of ethylene oxide with sorbitan fatty acid esters such as the TWEEN™ surfactants (ICI). The nonionic synthetic organic detergents generally are the condensation products of an organic aliphatic or alkyl aromatic hydrophobic compound and hydrophilic ethylene oxide groups. Practically any hydrophobic compound having a carboxy, hydroxy, amido, or amino group with a free hydrogen attached to the nitrogen can be condensed with ethylene oxide or with the polyhydration product thereof, polyethylene glycol, to form a water-soluble nonionic detergent. Further, the length of the polyethenoxy chain can be adjusted to achieve the desired balance between the hydrophobic and hydrophilic elements.

The nonionic surfactant class includes the condensation products of a higher alcohol (e.g., an alkanol containing about 8 to 8 carbon atoms in a straight or branched chain configuration) condensed with about 5 to 30 moles of ethylene oxide, for example, lauryl or myristyl alcohol condensed with about 16 moles of ethylene oxide (EO), tridecanol condensed with about 6 to moles of EO, myristyl alcohol condensed with about 10 moles of EO per mole of myristyl alcohol, the condensation product of EO with a cut of coconut fatty alcohol containing a mixture of fatty alcohols with alkyl chains varying from 10 to about 14 carbon atoms in length and wherein the condensate contains either about 6 moles of EO per mole of total alcohol or about 9 moles of EO per mole of alcohol and tallow alcohol ethoxylates containing 6 EO to 11 EO per mole of alcohol.

In one example, the nonionic surfactants may include the NEODOL™ ethoxylates (Shell Co.), which are higher aliphatic, primary alcohol containing about 9-15 carbon atoms, such as C₉-C₁₁ alkanol condensed with 2.5 to 10 moles of ethylene oxide (NEODOL™ 91-2.5 OR-5 OR-6 OR-8), C₁₂₋₁₃ alkanol condensed with 6.5 moles ethylene oxide (NEODOL™ 23-6.5), C₁₂₋₁₅ alkanol condensed with 7 moles ethylene oxide (NEODOL™ 25-7), C₁₂₋₁₅ alkanol condensed with 12 moles ethylene oxide (NEODOL™ 25-12), C₁₄₋₁₅ alkanol condensed with 13 moles ethylene oxide (NEODOL™ 45-13), and the like.

Additional satisfactory water-soluble alcohol ethylene oxide condensates may be the condensation products of a secondary aliphatic alcohol containing 8 to 18 carbon atoms in a straight or branched chain configuration condensed with 5 to 30 moles of ethylene oxide. Examples of commercially available nonionic detergents of the foregoing type arc C₁₁-C₁₅ secondary alkanol condensed with either 9 EO (TERGITOL™ 15-S-9) or 12 EO (TERGITOL™ 15-S-12) marketed by Dow Chemical.

Other suitable nonionic surfactants include the polyethylene oxide condensates of one mole of alkyl phenol containing from about 8 to 18 carbon atoms in a straight- or branched chain alkyl group with about 5 to 30 moles of ethylene oxide. Specific examples of alkyl phenol ethoxylates include, but are not limited to, nonyl phenol condensed with about 9.5 moles of EO per mole of nonyl phenol, dinonyl phenol condensed with about 12 moles of EO per mole of phenol, dinonyl phenol condensed with about 15 moles of EO per mole of phenol and di-isoctylphenol condensed with about 15 moles of EO per mole of phenol. Commercially available nonionic surfactants of this type include IGEPAL™ CO-630 (nonyl phenol ethoxylate) marketed by GAF Corporation.

Also among the satisfactory nonionic surfactants are the water-soluble condensation products of a C₈-C₂₀ alkanol with a mixture of ethylene oxide and propylene oxide wherein the weight ratio of ethylene oxide to propylene oxide is from 2.5:1 to 4:1, preferably 2.8:1 to 3.3:1, with the total of the ethylene oxide and propylene oxide (including the terminal ethanol or propanol group) being from 60-85%, preferably 70-80%, by weight. Such detergents are commercially available from BASF and a particularly preferred detergent is a C₁₀-C₁₆ alkanol condensate with ethylene oxide and propylene oxide, the weight ratio of ethylene oxide to propylene oxide being 3:1 and the total alkoxy content being about 75% by weight.

Condensates of 2 to 30 moles of ethylene oxide with sorbitan mono- and tri-C₁₀-C₂₀ alkanoic acid esters having a HLB of 8 to 15 also may be employed as the nonionic detergent ingredient in the described composition. These surfactants are well known and are available from Imperial Chemical Industries under the TWEEN™ trade name. Suitable surfactants include, but are not limited to, polyoxyethylene (4) sorbitan monolaurate, polyoxyethylene (4) sorbitan monostearate, polyoxyethylene (20) sorbitan trioleate and polyoxyethylene (20) sorbitan tristearate.

Other suitable water-soluble nonionic surfactants are marketed under the trade name PLURONIC™ The compounds are formed by condensing ethylene oxide with a hydrophobic base formed by the condensation of propylene oxide with propylene glycol. The molecular weight of the hydrophobic portion of the molecule is of the order of 950 to 4000 and preferably 200 to 2,500. The addition of polyoxyethylene radicals to the hydrophobic portion tends to increase the solubility of the molecule as a whole so as to make the surfactant water-soluble. The molecular weight of the block polymers varies from 1,000 to 15,000 and the polyethylene oxide content may comprise 20% to 80% by weight. Preferably, these surfactants will be in liquid form and satisfactory surfactants are available as grades L 62 and L 64.

The composition may include from 0.1 to 5 wt %, from 0.1 to 4 wt %, from 0.1 to 3 wt %, from 0.1 to 2 wt %, from 0.1 to 1 wt %, from 0.1 to 0.5 wt %, from 0.1 to 0.3 wt %, from 0.3 to 5 wt %, from 0.3 to 4 wt %, from 0.3 to 3 wt %, from 0.3 to 2 wt %, from 0.5 to 5 wt %, from 0.5 to 4 wt %, from 0.5 to 3 wt %, from 0.5 to 2 wt %, from 1 to 5 wt %, from 1 to 4 wt %, from 1 to 3 wt %, from 1 to 2 wt %, from 1 to 1.5 wt %, or any values within the foregoing ranges or any ranges created thereby, of active nonionic surfactant.

The composition may include anionic surfactants. Anionic surfactants include, but are not limited to, those surface-active or detergent compounds that contain an organic hydrophobic group containing generally 8 to 26 carbon atoms or generally 10 to 18 carbon atoms in their molecular structure and at least one water-solubilizing group selected from sulfonate, sulfate, and carboxylate so as to form a water-soluble detergent. Usually, the hydrophobic group will comprise a C₈-C₂₂ alkyl, or acyl group. Such surfactants are employed in the form of water-soluble salts and the salt-forming cation usually is selected from sodium, potassium, ammonium, magnesium and mono-, di- or tri-C₂-C₃ alkanolammonium, with the sodium, magnesium and ammonium cations again being the usual ones chosen.

The anionic surfactants that may be used in the composition are water soluble and include, but are not limited to, the sodium, potassium, ammonium, and ethanolammonium salts of linear C₈-C₁₆ alkyl benzene sulfonates, alkyl ether carboxylates, C₁₀-C₂₀ paraffin sulfonates, C₈-C₂₅ alpha olefin sulfonates, C₈-C₁₈ alkyl sulfates, alkyl ether sulfates and mixtures thereof.

The paraffin sulfonates (also known as secondary alkane sulfonates) may be monosulfonates or di-sulfonates and usually are mixtures thereof, obtained by sulfonating paraffins of 10 to 20 carbon atoms. Commonly used paraffin sulfonates are those of C12-18 carbon atoms chains, and more commonly they are of C14-17 chains. Such compounds may be made to specifications and desirably the content of paraffin sulfbnates outside the C14-17 range will be minor and will be minimized, as will be any contents of di- or poly-sulfonates. Examples of paraffin sulfonates include, but are not limited to HOSTAPUR™ SAS30, SAS 60, SAS 93 secondary alkane sulfonates from Clariant, and BIO-TERGE™ surfactants from Stepan, and CAS No. 68037-49-0.

Pareth sulfate surfactants can also be included in the composition. The pareth sulfate surfactant is a salt of an ethoxylated C₁₀-C₁₆ pareth sulfate surfactant having 1 to 30 moles of ethylene oxide. In some examples, the amount of ethylene oxide is 1 to 6 moles, and in other examples it is 2 to 3 moles, and in another example it is 2 moles. In one example, the pareth sulfate is a C₁₂-C₁₃ pareth sulfate with 2 moles of ethylene oxide. An example of a pareth sulfate surfactant is STEOL™ 23-2S/70 from Stepan, or (CAS No. 68585-34-2).

Examples of suitable other sulfonated anionic detergents are the well known higher alkyl mononuclear aromatic sulfonates, such as the higher alkylbenzene sulfonates containing 9 to 18 or preferably 9 to 16 carbon atoms in the higher alkyl group in a straight or branched chain, or C₈₋₁₅ alkyl toluene sulfonates. In one example, the alkylbenzene sulfonate is a linear alkylbenzene sulfonate having a higher content of 3-phenyl (or higher) isomers and a correspondingly lower content (well below 50%) of 2-phenyl (or lower) isomers, such as those sulfonates wherein the benzene ring is attached mostly at the 3 or higher (for example 4, 5, 6 or 7) position of the alkyl group and the content of the isomers in which the benzene ring is attached in the 2 or 1 position is correspondingly low. Materials that can be used are found in U.S. Pat. No. 3,320,174, especially those in which the alkyls are of 10 to 13 carbon atoms.

Other suitable anionic surfactants are the olefin sulfonates, including long-chain alkene sulfonates, long-chain hydroxyalkane sulfonates or mixtures of alkene sulfonates and hydroxyalkane sulfonates. These olefin sulfonate detergents may be prepared in a known manner by the reaction of sulfur trioxide (SO₃) with long-chain olefins containing 8 to 25, preferably 12 to 21 carbon atoms and having the formula RCH═CHR₁ where R is a higher alkyl group of 6 to 23 carbons and Ri is an alkyl group of 1 to 17 carbons or hydrogen to form a mixture of sultones and alkene sulfonic acids which is then treated to convert the sultones to sulfonates. In one example, olefin sulfonates contain from 14 to 16 carbon atoms in the R alkyl group and are obtained by sulfonating an a-olefin.

Examples of satisfactory anionic sulfate surfactants are the alkyl sulfate salts and the and the alkyl ether polyethenoxy sulfate salts having the formula R(OC₂H₄)ₙOSO₃M wherein n is 1 to 12, or 1 to 5, and R is an alkyl group having about 8 to about 18 carbon atoms, or 12 to 15 and natural cuts, for example, C₁₂₋₁₄ or C₁₂₋₁₆ and M is a solubilizing cation selected from sodium, potassium, ammonium, magnesium and mono-, di- and triethanol ammonium ions. The alkyl sulfates may be obtained by sulfating the alcohols obtained by reducing glycerides of coconut oil or tallow or mixtures thereof and neutralizing the resultant product.

The ethoxylated alkyl ether sulfate may be made by sulfating the condensation product of ethylene oxide and C₈₋₁₈ alkanol, and neutralizing the resultant product. The ethoxylated alkyl ether sulfates differ from one another in the number of carbon atoms in the alcohols and in the number of moles of ethylene oxide reacted with one mole of such alcohol. In one example, alkyl ether sulfates contain 12 to 15 carbon atoms in the alcohols and in the alkyl groups thereof, e.g., sodium myristyl (3 EO) sulfate.

Ethoxylated C₈₋₁₈ alkylphenyl ether sulfates containing from 2 to 6 moles of ethylene oxide in the molecule may also be suitable for use in the composition. These detergents can be prepared by reacting an alkyl phenol with 2 to 6 moles of ethylene oxide and sulfating and neutralizing the resultant ethoxylated alkylphenol.

Other suitable anionic detergents are the C₉-C₁₅ alkyl ether polyethenoxylcarboxylates having the structural formula R(OC₂H₄)ₙOX COOH wherein n is a number from 4 to 12, preferably 6 to 11 and X is selected from the group consisting of CH₂, C(O) R₁ and wherein Riis a C₁-C₃ alkylene group. Types of these compounds include, but are not limited to, C₉-C₁₁ alkyl ether polyethenoxy (7-9) C(O)CH₂CH₂COOH, C₁₃-C₁₅ alkyl ether polyethenoxy (7-9) and C₁₀-C₁₂ alkyl ether polyethenoxy (5-7) CH₂COOH. These compounds may be prepared by condensing ethylene oxide with appropriate alkanol and reacting this reaction product with chloracetic acid to make the ether carboxylic acids as shown in U.S. Pat. No. 3,741,911 or with succinic anhydride or phtalic anhydride.

The anionic surfactant may include dioctyl sodium sulfosuccinate.

The composition may include from 0 to 5 wt %, from 0 to 2 wt %, from 0 to 1 wt %, from 0 to 0.5 wt %, from 0 to 0.2 wt %, from 0.01 to 5 wt %, from 0.01 to 2 wt %, from 0.01 to 1 wt %, from 0.01 to 0.5 wt %, from 0.01 to 0.2 wt %, from 0.05 to 5 wt %, from 0.05 to 2 wt %, from 0.05 to 1 wt %, from 0.05 to 0.5 wt %, from 0.05 to 0.2 wt %, from 0.1 to 5 wt %, from 0.1 to 2 wt %, from 0.1 to 1 wt %, from 0.1 to 0.5 wt %, from 0.1 to 0.2 wt %, about 0.15 wt %, or any values within the foregoing ranges or any ranges created thereby, of active anionic surfactant.

The composition may include from 0.1 to 10 wt %, from 0.1 to 5 wt %, from 0.1 to 4 wt %, from 0.1 to 3 wt %, from 0.1 to 2 wt %, from 0.1 to 1 wt %, from 0.1 to 0.5 wt %, from 0.1 to 0.3 wt %, from 0.3 to 5 wt %, from 0.3 to 4 wt %, from 0.3 to 3 wt %, from 0.3 to 2 wt %, from 0.5 to 5 wt %, from 0.5 to 4 wt %, from 0.5 to 3 wt %, from 0.5 to 2 wt %, from 1 to 5 wt %, from 1 to 4 wt %, from 1 to 3 wt %, from 1 to 2 wt %, from 1 to 1.5 wt %, or any values within the foregoing ranges or any ranges created thereby, of active surfactant.

### Additional Optional Ingredients

Additional optional ingredients may be included to provide added effect or to make the product more attractive. Such ingredients include, but are not limited to, propellants, buffers, fragrances, abrasive agents, disinfectants, radical scavengers, bleaches, chelating agents, antibacterial agents/preservatives, optical brighteners, hydrotropes, or combinations thereof.

The propellant may include compressed gas, such as nitrogen and air, condensable gas, such as hydro-fluorinated olefins (HFO), including as trans-1,3,3,3-tetrafluoroprop-1-ene, and mixtures thereof. Propellants listed in the US Federal Register 49 CFR 1.73.115, Class 2, Division 2.2 may be acceptable. The propellant may be condensable. A condensable propellant, when condensed, may provide the benefit of a flatter depressurization curve at the vapor pressure, as composition is depleted during usage. A condensable propellant may provide the benefit that a greater volume of gas may be placed into the container at a given pressure. Generally, the highest pressure occurs after the spray dispenser is charged with the composition but before the first dispensing of that composition by the user.

The composition may include a buffering agent. The buffering agent may be an acidic buffering agent. The buffering agent may be a dibasic or tribasic acid (such as phosphoric acid), carboxylic acid, a dicarboxylic acid, or a tricarboxylic acid. The carboxylic acid may be, for example, citric acid, polyacrylic acid, or maleic acid. The acid may be sterically stable. The acid may be used in the composition for maintaining the desired pH. The composition may have a pH from about 4 to about 11, alternatively from about 4 to about 9, alternatively from about 4 to about 6.9, alternatively about 4 to about 7.

The composition may include from 0.1 to 2 wt %, from 0.1 to 1 wt %, from 0.1 to 0.7 wt %, from 0.3 to 2 wt %, from 0.3 to 1 wt %, from 0.3 to 0.7 wt %, about 0.5 wt % or any values within the foregoing ranges or any ranges created thereby, of active buffer.

The buffer system may comprise one or more buffering agents selected from the group consisting of: citric acid, maleic acid, polyacrylic acid, and combinations thereof. It has been found that buffer systems that include a buffering agent selected from the group consisting of: citric acid, maleic acid, polyacrylic acid, and combinations thereof provide stable compositions with prolonged shelf life.

The buffer system may comprise citric acid and sodium citrate. It has been found that buffer systems comprising citric acid and sodium citrate provide stable compositions with a prolonged shelf life.

Other suitable buffering agents for the compositions include biological buffering agents. Some examples are nitrogen-containing materials, sulfonic acid buffers like 3-(N-morpholino) propanesulfonic acid (MOPS) or N-(2-Acetamido)-2-aminoethanesulfonic acid (ACES), which have a near neutral 6.2 to 7.5 pKa and provide adequate buffering capacity at a neutral pH. Other examples are amino acids such as lysine or lower alcohol amines like mono-, di-, and tri-ethanolamine or methyldiethanolamine or derivatives thereof. Other nitrogen-containing buffering agents are tri (hydroxymethyl)amino methane (HOCH2)3CNH3 (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol N,N′-tetra-methyl-1,3-diamino-2-propanol, N,N-bis(2-hydroxyethyl)glycine (bicine) and N-tris(hydroxymethyl)methyl glycine (tricine). Mixtures of any of the above are also acceptable.

The composition may include an effective amount of a compound for reducing microbes in the air or on inanimate surfaces. Antimicrobial compounds are effective on gram negative and gram positive bacteria and fungi typically found on indoor surfaces that have contacted human skin or pets such as couches, pillows, pet bedding, and carpets. Such microbial species include Klebsiella pneumoniae, Staphylococcus aureus, Aspergillus niger, Klebsiella pneumoniae, Steptococcus pyogenes, Salmonella choleraesuis, Escherichia coli, Trichophyton mentagrophytes, and Pseudomonas aeruginosa. The antimicrobial compounds may also effective on viruses such H1-N1, Rhinovirus, Respiratory Syncytial, Poliovirus Type 1, Rotavirus, Influenza A, Herpes simplex types 1 & 2, Hepatitis A. and Human Coronavirus.

Antimicrobial compounds suitable in the composition can be any organic material which will not cause damage to fabric appearance (e.g., discoloration, coloration such as yellowing, bleaching). Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof.

The composition may include a quaternary compound. Examples of commercially available quaternary compounds suitable for use in the composition is BARQUAT® available from Arxada; and didecyl dimethyl ammonium chloride quat under the trade name BARDAC® 2250 from Arxada Corporation.

The antimicrobial compound, if present, may be present in an amount from about 500 ppm to about 7000 ppm, alternatively from about 1000 ppm to about 5000 ppm, alternatively from about 1000 ppm to about 3000 ppm, alternatively from about 1400 ppm to about 2500 ppm, by weight of the composition.

The composition may include a preservative. The preservative may be included in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase the shelf-life of the composition.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diaol materials, isothiazolinones, quaternary compounds, benzoates, low molecular weight alcohols, dehydroacetic acid, phenyl and phenoxy compounds, or mixtures thereof.

Non-limiting examples of water-soluble preservatives include a mixture of about 77% 5-chloro-2-methyl-4-isothiazolin-3-one and about 23% 2-methyl-4-isothiazolin-3-one, a broad spectrum preservative available as a 1.5% freshening solution under the trade name Kathon® CG by LANXESS; 5-bromo-5-nitro-1,3-dioxane, available under the tradename Bronidox L® from BASF; 2-bromo-2-nitropropane-1,3-diol, available under the trade name Bronopol® from Jan Dekker; 1, l′-hexamethylene bis(5-(p-chlorophenyl) biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl) 2,5-dioxo-4-imidazolidinyl]-N,N′-bis(hydroxy-methyl) urea, commonly known as diazolidinyl urea, available under the trade name Germall® II from Ashland, Inc.; N,N″-methylenebis {N′-[1-(hydroxymethyl)-2,5-dioxo-4-imidazolidinyl]urea}, commonly known as imidazolidinyl urea, available, e.g., under the trade name Abiol® from 3V-Sigma; Unicide U-13® from Brulin; Germall 115® from Ashland; polymethoxy bicyclic oxazolidine, available under the trade name Nuosept® C from Arxada-Troy Corp; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from LANXESS.

The preservative, if present, may be present at a level of from about 0.0001% to about 0.5%, alternatively from about 0.0002% to about 0.2%, alternatively from about 0.0003% to about 0.1%, by weight of the composition.

The preservative may not be present in the composition of the present disclosure.

The composition may include a wetting agent that provides a low surface tension that permits the composition to spread readily and more uniformly on hydrophobic surfaces like polyester and nylon. The spreading of the composition also allows it to dry faster, so that the treated material is ready to use sooner. Furthermore, a composition containing a wetting agent may penetrate hydrophobic, oily soil better for improved malodor neutralization. A composition containing a wetting agent may also provide improved “in-wear” electrostatic control. For concentrated compositions, the wetting agent facilitates the dispersion of many actives such as antimicrobial actives and perfumes in the concentrated compositions.

Non-limiting examples of wetting agents include block copolymers of ethylene oxide and propylene oxide. Suitable block polyoxyethylene-polyoxypropylene polymeric surfactants include those based on ethylene glycol, propylene glycol, glycerol, trimethylolpropane and ethylenediamine as the initial reactive hydrogen compound. Polymeric compounds made from a sequential ethoxylation and propoxylation of initial compounds with a single reactive hydrogen atom, such as C₁₂₋₁₈ aliphatic alcohols, are not generally compatible with the cyclodextrin. Certain of the block polymer surfactant compounds designated Pluronic® and Tetronic® by the BASF-Wyandotte Corp., Wyandotte, Michigan, are readily available.

Non-limiting examples of cyclodextrin-compatible wetting agents of this type are described in U.S. Pat. No. 5,714,137 and include the Silwet® surfactants available from Momentive Performance Chemical, Albany, New York. Exemplary Silwet surfactants are as follows:

| Name | Average MW |
|---|---|
| L-7608 | 600 |
| L-7607 | 1,000 |
| L-77 | 600 |
| L-7605 | 6,000 |
| L-7604 | 4,000 |
| L-7600 | 4,000 |
| L-7657 | 5,000 |
| L-7602 | 3,000; |

and mixtures thereof.

The total amount of surfactants (e.g. solubilizer, wetting agent) present in the composition may be from 0% to about 3% or no more than 3%, alternatively from 0% to about 2% or no more than 2%, alternatively from 0% to about 1.5% or no more than 1.5%, alternatively from 0% to about 1% or no more than 1%, alternatively from 0% to about 0.5% or no more than 0.5%, alternatively from 0% to 0.3% or no more than about 0.3%, or any values within the foregoing ranges or any ranges created thereby, active by weight of the composition. Compositions with higher concentrations may make fabrics susceptible to soiling and/or leave unacceptable visible stains on fabrics as the solution evaporates.

The composition can have any desired pH. In some examples, the composition is neutral to basic. The composition may have a pH of less than 10. The composition may have a pH between 3 to 10, such as, for example, a pH between 3 and 7, a pH between 4 and 6, a pH between 4.5 and 5.5, a pH of about 5, or any values within the foregoing ranges or any ranges created thereby. It is contemplated that, at a pH as described, the buffer (e.g. citric acid) may be more effective at buffering, as opposed to a differing pH, and the buffer may react with/neutralize amine malodors at a pH as described.

### EXAMPLES

### Example 1: Method of Making a Composition in Accordance with the Present Disclosure

### Preparation of the Eutectic Solvent:

TABLE 1
Eutectic solvent composition with 5:1
molar ratio of Lactic Acid:Fructose

| | Material | Active | Formula |
|---|---|---|---|
| Material | Activity | wt % | wt % |
| DL- Lactic Acid (Thermo Fisher Scientific) | 90.00% | 66.18% | 73.53% |
| D-Fructose (VWR) | 99.00% | 26.21% | 26.47% |

1. Using an analytical balance to weigh materials, add natural deep eutectic solvent components (e.g. lactic acid and fructose) to a glass beaker at the specified molar ratio (e.g. 5:1 lactic acid:fructose) on a % active basis. Solid forms/powders can be added slowly during heating & mixing step (2-3), if desired, to enable them to be blended in more easily.
2. Place beaker on a hotplate fitted with a temperature monitoring probe to control the temperature of the mixture in the beaker. Place temperature probe so that it is immersed in the mixture in the beaker. Set the setpoint of the temperature probe to 60 C according to manufacturer's instructions for the hotplate.
3. Use an IKA overhead mixer fitted with a dispersion impeller mixing blade to stir the mixture at ˜500 rpm. Thoroughly blend the components together.
4. Once mixture reaches a temperature of ˜60 C, continue maintaining this temperature while stirring for a total of ˜60 minutes.
5. Remove mixture from hotplate and cool to room temperature. Mixture should appear as a homogeneous liquid after cooling to 20-25° C.
   Preparation of a Composition in Accordance with the Present Disclosure:

TABLE 2
Base formula 1

| | | Raw Material | Active | Formula |
|---|---|---|---|---|
| Material | Function | Activity | wt % | wt % |
| Perfume | Fragrance | 100.00% | 1.51% | 1.51% |
| Eutectic Solvent | Solvent | 100.00% | X % | X % |
| Hydrogenated castor oil | Nonionic | 90.00% | 1.30% | 1.444% |
| (HCO) - Emulan ELH-60 | Surfactant | | | |
| available from BASF | | | | |
| Deionized Water | Solvent | 100.00% | balance | balance |
| Citric Acid Solution - | Buffer | 50.00% | 0.184% | 0.368% |
| available from Univar | | | | |
| Solutions | | | | |
| Trisodium Citrate Dihydrate - | Buffer | 88.00% | 0.264% | 0.300% |
| available from Archer Daniels | | | | |
| Midland Company | | | | |
| Dioctyl Sodium | Anionic | 71.00% | 0.142% | 0.20% |
| Sulfosuccinate (DOSS) - | Surfactant | | | |
| Aerosol OT-70 Anionic | | | | |
| surfactant available from | | | | |
| Solvay | | | | |
| Hydroxypropyl Beta | Malodor | 40.00% | 0.15% | 0.375% |
| Cyclodextrin - Cavasol W7 | Reducer | | | |
| HP TL available from Wacker | | | | |
| 1,2-benzisothiazolin-3-one | Preservative | 19.00% | 0.0044% | 0.023% |
| (BIT) - Koralone B-119 | | | | |
| available from LANXESS | | | | |
| Sodium hydroxide - Formosa | pH adjustment | 50.00% | As needed | As needed |
| Plastics Corporation | | | for pH | for pH |

6. Perfume Premix. Weigh the specified quantity of perfume components, the eutectic solvent prepared in steps 1-5, and hydrogenated castor oil into a suitably sized beaker. Add a stir bar to the mixture and place on a stir plate. Stir mixture at moderate speed for at least 15 minutes, or until ingredients are mixed thoroughly. Cover beaker and continue stirring mixture at room temperature (20-25° C.) until it is used in step 11 below. Premix should be used within 4 hours after making.
7. In a separate glass beaker, add the specified amount of deionized water.
8. Use an IKA overhead mixer fitted with a pitched blade impeller (of suitable size to thoroughly blend the mixture) to begin stirring water at a speed which is adequate to form a mixing vortex in the center of the liquid mixture, but does not cause aeration of the liquid.
9. Using an analytical balance to weigh components, add the following components to the water in the following order. Continue overhead mixing and stir mixture for ˜5 minutes after addition of each component before adding the next component. Order of addition: citric acid, then trisodium citrate dihydrate, then, DOSS.
10. For formulation(s) containing Lactic acid and Fructose added as separate components, add the specified amount of lactic acid, and then fructose to the formula. Stir during and for ˜5 minutes after each addition, or until all the fructose powder is completely dissolved.
11. Add the specified amount of the perfume premix prepared in step 6. Stir during and for ˜5 minutes after addition.
12. Add the specified amount of the following components to the mixture while continuing to stir mixture. Stir mixture for ˜5 minutes after addition of each component before adding the next component. Order of addition: hydroxypropyl beta cyclodextrin, then 1,2-benzisothiazolin-3-one (BIT).
13. Once all ingredients are added, continue stirring mixture for ˜5 minutes.
14. While continuing to stir mixture, use a calibrated pH probe to measure the pH of the mixture. If the pH is below 5.0, add 50% sodium hydroxide solution dropwise while continuing to stir and monitor the pH until the pH is 4.75-5.10.
15 Using a Hach 2100Q Portable Turbidimeter (Catalog #2100Q01) per manufacturer's instructions, measure the turbidity of the solution.
   Packaging of Formulations into Aerosols for Stability Assessment:
16. Obtain a transparent polyethylene terephthalate bottle suitable for aerosol packaging. Add enough finished formula to the bottle to fill it ½ to ¾ of the way full.
17. Use a crimping device to crimp/attach a valve stem assembly to the top of the bottle to make the bottle air-tight.
18. Pressurize the bottle to ˜120-150 psi using nitrogen gas.
19. Store bottles containing finished formulas at the following constant temperature and/or temperature/humidity conditions: 5° C./60% relative humidity, 25° C./60% relative humidity, 50° C. and 3 cycles of freeze/thaw (24 hours at −18° C./24 hours at 15° C./60% relative humidity). Observe the visual appearance of the formula over time.

### Example 2: Stability Assessments for Eutectic Solvents Made Via the Process Above (Steps 1-5) in Example 1 and Added to the Base Formula 1 Via the Process Above in Example 1 (Steps 6-19)

Initial appearance and turbidity were collected on formulas which were made and then stored in glass jars. Stability over time was measured on finished formulas stored in aerosol bottles.

TABLE 3
Formula compositions where the eutectic solvent ratios are molar ratios

| Formula | Composition |
|---|---|
| A | Base Formula 1 with no eutectic solvent and with 0.8 wt % active HCO (instead of |
| | 1.3 wt % active HCO) |
| 1 | Base Formula 1 with X = 2 wt % active Eutectic Solvent (5:1 Lactic Acid:Fructose) |
| | and with 0.8 wt % active HCO (instead of 1.3 wt % active HCO) |
| B | Base Formula 1 with no eutectic solvent |
| 2 | Base Formula 1 with X = 1 wt % active Eutectic Solvent (5:1 Lactic Acid:Fructose) |
| 3 | Base Formula 1 with X = 1 wt % active Eutectic Solvent (5:1 Lactic Acid:Fructose) |
| C | Base Formula 1 with no eutectic solvent and with 1 wt % active 5:1 Lactic |
| | Acid:Fructose added in bulk (lactic acid & fructose added as separate components |
| | into the bulk formula per step 10 of the Example 1 making process) |
| D | Base Formula 1 with no eutectic solvent and with no anionic surfactant (DOSS) |
| 4 | Base Formula 1 with X = 1 wt % active Eutectic Solvent (5:1 Lactic Acid:Fructose) |
| | and with no anionic surfactant (DOSS) |
| 5 | Base Formula 1 with X = 1 wt % active Eutectic Solvent (5:1 Lactic Acid:Sorbitol) |
| 6 | Base Formula 1 with X = 1 wt % active Eutectic Solvent (3:1 Lactic Acid:Fructose) |
| 7 | Base Formula 1 with X = 1 wt % active Eutectic Solvent (8:1 Lactic Acid:Fructose) |
| 8 | Base Formula 1 with X = 1 wt % active Eutectic Solvent (5:1 Lactic Acid:Glucose) |
| 9 | Base Formula 1 with X = 1 wt % active Eutectic Solvent (8:1 Lactic Acid:Sucrose) |
| 10 | Base Formula 1 with X = 1 wt % active Eutectic Solvent (5:0.5:0.5 Lactic |
| | Acid:Glucose:Xylose) |
| 11 | Base Formula 1 with X = 1 wt % active Eutectic Solvent (1:1:12 Citric |
| | Acid:Glucose:Water) |
| 12 | Base Formula 1 with X = 2 wt % active Eutectic Solvent (5:1 Lactic Acid:Fructose) |
| | and with 1.6 wt % active HCO (instead of 1.3 wt % active HCO) and with 1.96 wt % |
| | active perfume (instead of 1.51 wt % active perfume) |

TABLE 4
Turbidity and Appearance Data. Turbidity is measured
in Nephelometric Turbidity Units (NTU).

| | Turbidity | | Was a |
|---|---|---|---|
| | (NTU) @ | | Precipitate |
| Formula | 1 week old | Initial Appearance | Present? |
| A | 909 | Opaque with fine white | Yes |
| | | precipitate around top edge | |
| 1 | 84.8 | Hazy, homogeneous | No |
| B | 198 | Hazy with fine white precipitate | Yes |
| | | around top edge | |
| 2 | 57 | Hazy, homogeneous | No |
| 3 | 67.1 | Slightly hazy | No |
| C | 138 | Opaque with fine white | Yes |
| | | precipitate around top edge | |
| D | 374 | Cloudy with fine white | Yes |
| | | precipitate around top edge | |
| 4 | 146 | Very hazy, homogeneous | No |
| 5 | 66.6 | Slightly hazy | No |
| 6 | 74.1 | Slightly hazy | No |
| 7 | 60.3 | Slightly hazy | No |
| 8 | 64.1 | Slightly hazy | No |
| 9 | 73.8 | Slightly hazy | No |
| 10 | 62.1 | Slightly hazy | No |
| 11 | 62.8 | Slightly hazy | No |
| 12 | 63.3 | Slightly hazy | No |

Both the turbidity and initial appearance data support that formulations containing the eutectic solvent formed via the process outlined in Example 1 (Formulas 1-12) are less turbid and have a homogeneous initial appearance without opacity or precipitation as compared to Formulas A-D. In comparison, the formulas without the eutectic solvent (Formulas A, B, and D) and the formula where the eutectic solvent materials were added as separate components without first forming the eutectic mixture (Formula C) exhibited stability failure by forming precipitate and having high turbidity.

In examples, the compositions in accordance with the present disclosure may have a turbidity at 1 week of from 10 to 150 NTU, from 10 to 100 NTU, from 10 to 90 NTU, from 10 to 80 NTU, from 30 to 130 NTU, from 30 to 100 NTU, from 30 to 90 NTU, from 30 to 80 NTU, from 50 to 130 NTU, from 50 to 100 NTU, from 50 to 90 NTU, from 50 to 80 NTU, or any values within the foregoing ranges or any ranges created thereby.

### Example 3: Method of Making a Composition Including Unmodified Beta-Cyclodextrin (BCD) in Accordance with the Present Disclosure

### Preparation of the Eutectic Solvent:

TABLE 5
Eutectic solvent mixture with 5:1 molar ratio of Lactic
Acid:Fructose euytectic solvent and betacyclodextrin

| | Raw | Active | |
|---|---|---|---|
| | Material | wt % | Formula |
| Material | Activity | (Z %) | wt % |
| DL- Lactic Acid (Thermo Fisher Scientific) | 90.00% | 58.19% | 64.66% |
| D-Fructose (VWR) | 99.00% | 23.05% | 23.28% |
| Unmodified Beta-cyclodextrin (Wacker) | 86.00% | 10.38% | 12.07% |

### Preparation of Eutectic Solvent Mixture Including Eutectic Solvent and Unmodified Beta Cyclodextrin:

1. Using an analytical balance to weigh materials, add natural deep eutectic solvent components (lactic acid and fructose (or HBA and HBD used)) to a glass beaker at the specified molar ratio (e.g. 5:1 lactic acid:fructose) on a % active basis. Solid forms/powders can be added slowly during heating & mixing step, if desired, to enable them to be blended in more easily.
2. Using the analytical balance to weigh materials, also add unmodified beta-cyclodextrin powder (Cavamax W7 available from Wacker) to the mixture at a Z active wt % (up to 20%) of the weight of the eutectic solvent mixture. Solid forms/powders can be added slowly during heating & mixing step, if desired, to enable them to be blended in more easily.
3. Place beaker on a hotplate fitted with a temperature monitoring probe to control the temperature of the mixture in the beaker. Place temperature probe so that it is immersed in the mixture in the beaker. Set the setpoint of the temperature probe to 60° C. according to manufacturer's instructions for the hotplate.
4. Use an IKA overhead mixer fitted with a dispersion impeller mixing blade to stir the mixture at ˜500 rpm. Thoroughly blend the components together.
5. Once mixture reaches a temperature of 60° C., continue maintaining this temperature while stirring for a total of 60 minutes.
6. Remove mixture from hotplate and cool to room temperature. Mixture should appear as a homogeneous liquid after cooling to 20-25° C.
   Preparation of a Composition Including Eutectic Solvent Having Unmodified BCD in Accordance with the Present Disclosure:

TABLE 6
Base formula 2

| | | Raw | | |
|---|---|---|---|---|
| | | Material | Active | Formula |
| Material | Function | Activity | wt % | wt % |
| Perfume | Fragrance | 100.00% | 1.51% | 1.51% |
| Hydrogenated castor oil (HCO) - | Nonionic | 90.00% | 1.80% | 2% |
| Emulan ELH-60 available from | Surfactant | | | |
| BASF | | | | |
| Deionized Water | Solvent | 100.00% | balance | balance |
| Ethanol - available from Grain | Solvent | 93.00% | 0% | 0% |
| Processing Corporation | | | | |
| Citric Acid Solution - available | Buffer | 50.00% | 0.184% | 0.368% |
| from Univar Solutions | | | | |
| Trisodium Citrate Dihydrate - | Buffer | 88.00% | 0.264% | 0.300% |
| available from Archer Daniels | | | | |
| Midland Company | | | | |
| Dioctyl Sodium Sulfosuccinate | Anionic | 71.00% | 0.142% | 0.20% |
| (DOSS) - Aerosol OT-70 Anionic | Surfactant | | | |
| surfactant available from Solvay | | | | |
| Eutectic Solvent (not including | Solvent | 100.00% | V % | V % |
| BCD) | | | | |
| Unmodified Beta Cyclodextrin | Malodor | 86.00% | W % | W % |
| (BCD) - Cavamax W7 available | Reducer | | | |
| from Wacker | | | | |
| Hydroxypropyl Beta Cyclodextrin | Malodor | 40.00% | Y % | Y % |
| (BCD) - Cavasol W7 HP TL | Reducer | | | |
| available from Wacker | | | | |
| 1,2-benzisothiazolin-3-one (BIT) - | Preservative | 19.00% | 0.0044% | 0.023% |
| Koralone B-119 available from | | | | |
| LANXESS | | | | |
| Sodium hydroxide - Formosa | pH | 50.00% | As needed | As needed |
| Plastics Corporation | adjustment | | for pH | for pH |

7. Perfume Premix. Weigh the specified quantity of perfume components and hydrogenated castor oil into a suitably sized beaker. Add a stir bar to the mixture and place on a stir plate. Stir mixture at moderate speed for at least 15 minutes, or until ingredients are mixed thoroughly. Cover beaker and continue stirring mixture at room temperature (20-25 C) until it is used in step 11 below. Premix should be used within 4 hours after making.
8. In a separate glass beaker, add the specified amount of deionized water.
9. Use an IKA overhead mixer fitted with a pitched blade impeller (of suitable size to thoroughly blend the mixture) to begin stirring water at a speed which is adequate to form a mixing vortex in the center of the liquid mixture, but does not cause aeration of the liquid.
10. Using an analytical balance to weigh components, add the following components to the water in the following order. Continue overhead mixing and stir mixture for ˜5 minutes after addition of each component before adding the next component. Order of addition: ethanol (if added), then buffer (e.g. citric acid solution), then trisodium citrate dihydrate, then DOSS.
11. Add the specified amount of the perfume premix prepared in step 7. Stir during and for ˜5 minutes after addition.
12. Add the specified amount of the following components to the mixture while continuing to stir mixture. Stir mixture for ˜5 minutes after addition of each component before adding the next component. Order of addition: Where one of (1) Eutectic Solvent mixture including Z active wt % unmodified betacyclodextrin; (2) hydroxypropyl betacyclodextrin without eutectic solvent; or (3) unmodified betacyclodextrin powder without eutectic solvent is added, then 1,2-benzisothiazolin-3-one (BIT).
13. Once all ingredients are added, continue stirring mixture for ˜5 minutes.
14. While continuing to stir mixture, use a calibrated pH probe to measure the pH of the mixture. If the pH is below 5.0, add 50% sodium hydroxide solution dropwise while continuing to stir and monitor the pH until the pH is 4.75-5.10.
15. Using a Hach 2100Q Portable Turbidimeter (Catalog #2100Q01) per manufacturer's instructions, measure the turbidity of the solution.

Packaging of formulations into aerosols for stability assessment. Formulations with an initial appearance having a precipitate and/or severe phase separation can skip steps 16-19 and have stability marked as “NA”.
16. Obtain a transparent polyethylene terephthalate bottle suitable for aerosol packaging. Add enough finished formula to the bottle to fill it ½ to ¾ of the way full.
17. Use a crimping device to crimp/attach a valve stem assembly to the top of the bottle to make the bottle air-tight.
18. Pressurize the bottle to ˜120-150 psi using nitrogen gas.
19. Store bottles containing finished formulas at the following constant temperature and/or temperature/humidity conditions: 5° C./60% relative humidity, 25° C./60% relative humidity, 50° C. and 3 cycles of freeze/thaw (24 hours at −18° C./24 hours at 15° C./60% relative humidity). Observe the visual appearance of the formula over time.

### Example 4: Stability Assessments for Formula Made Via the Process Above in Example 3

Initial appearance and turbidity were collected on formulas which were made and then stored in glass jars.

TABLE 7
Formula compositions where the eutectic solvent ratios are molar ratios

| | V (wt % active Eutectic | W (wt % active | Y (wt % active |
|---|---|---|---|
| Formula | Solvent) | Unmodified BCD) | HPBCD) |
| L | Base Formula 2 with no eutectic solvent and with HPBCD | | |
| | V = 0% | W = 0% | Y = 0.15 wt % active |
| | | | (0.38 wt % formula) |
| 21 | Base Formula 2 with eutectic solvent and with unmodified BCD | | |
| | V = 1.88 wt % active (5:1 | W = 0.12 wt % active | Y = 0% |
| | Lactic Acid:Fructose) | (0.14 wt % formula) | |
| | | (Z = 7% solvent mixture*) | |
| 22 | Base Formula 2 with eutectic solvent and with unmodified BCD | | |
| | V = 1.60 wt % active (5:1 | W = 0.15 wt % active | Y = 0% |
| | Lactic Acid:Fructose) | (0.17 wt % formula) | |
| | | (Z = 10% solvent mixture) | |
| 23 | Base Formula 2 with eutectic solvent and with unmodified BCD | | |
| | V = 0.72 wt % active (5:1 | W = 0.15 wt % active | Y = 0% |
| | Lactic Acid:Fructose) | (0.17 wt % formula) | |
| | | (Z = 20% solvent mixture) | |
| 24 | Base Formula 2 with eutectic solvent and with unmodified BCD | | |
| | V = 1.17 wt % active (5:1 | W = 0.24 wt % active | Y = 0% |
| | Lactic Acid:Fructose) | (0.28 wt % formula) | |
| | | (Z = 20% solvent mixture) | |
| 25 | Base Formula 2 with eutectic solvent and with unmodified BCD and with 5 wt % | | |
| | active Ethanol (instead of 0% ethanol) | | |
| | V = 1.88 wt % active (5:1 | W = 0.12 wt % active | Y = 0% |
| | Lactic Acid:Fructose) | (0.14 wt % formula) | |
| | | (Z = 7% solvent mixture) | |
| 26 | Base Formula 2 with eutectic solvent and with unmodified BCD and with 5 wt % | | |
| | active Ethanol (instead of 0% ethanol) | | |
| | V = 1.60 wt % active (5:1 | W = 0.15 wt % active | Y = 0% |
| | Lactic Acid:Fructose) | (0.17 wt % formula) | |
| | | (Z = 10% solvent mixture) | |
| 27 | Base Formula 2 with eutectic solvent and with unmodified BCD and with 5 wt % | | |
| | active Ethanol (instead of 0% ethanol) | | |
| | V = 0.72 wt % active (5:1 | W = 0.15 wt % active | Y = 0% |
| | Lactic Acid:Fructose) | (0.17 wt % formula) | |
| | | (Z = 20% solvent mixture) | |
| M | Base Formula 2 with no eutectic solvent and with HPBCD and with no anionic | | |
| | surfactant (DOSS) | | |
| | V = 0% | W = 0% | Y = 0.15 wt % active |
| | | | (0.38 wt % formula) |
| 28 | Base Formula 2 with eutectic solvent and unmodified BCD and with no anionic | | |
| | surfactant (DOSS) | | |
| | V = 1.28 wt % active (5:1 | W = 0.12 wt % active | Y = 0% |
| | Lactic Acid:Fructose) | (0.14 wt % formula) | |
| | | (Z = 10% solvent mixture) | |
| N | Base Formula 2 with no eutectic solvent and with unmodified BCD | | |
| | V = 0% | W = 0.15 wt % active | Y = 0% |
| | | (0.17 wt % formula) | |
| O | Base Formula 2 with no eutectic solvent and with HPBCD, 1.30 wt % active | | |
| | perfume (instead of 1.51 wt % active perfume), 2.10 wt % active HCO (instead of | | |
| | 1.80 wt % active HCO), 0.10 wt % active Sodium Polyacrylate Solution (KemEcal | | |
| | 142 PG available from Kemira Oyj) as the buffer instead of the citric acid solution, | | |
| | and a pH between 6.75-7.10 instead of 4.75-5.10 | | |
| | V = 0% | W = 0% | Y = 0.30 wt % active |
| | | | (0.75 wt % formula) |
| 29 | Base Formula 2 with eutectic solvent, unmodified BCD, 1.30 wt % active perfume | | |
| | (instead of 1.51 wt % active perfume), 2.10 wt % active HCO (instead of 1.80 wt % | | |
| | active HCO), 0.10% wt % active Sodium Polyacrylate Solution (KemEcal 142 PG | | |
| | available from Kemira Oyj) as the buffer instead of the citric acid solution, and a pH | | |
| | between 6.75-7.10 instead of 4.75-5.10 | | |
| | V = 3.18 wt % active (5:1 | W = 0.30 wt % active | Y = 0% |
| | Lactic Acid:Fructose) | (0.35 wt % formula) | |
| | | (Z = 10% solvent mixture) | |
| 30 | Base Formula 2 with eutectic solvent, unmodified BCD, 1.30 wt % active perfume | | |
| | (instead of 1.51 wt % active perfume), 2.10 wt % active HCO (instead of 1.80 wt % | | |
| | active HCO) and with 0.10 wt % active Sodium Polyacrylate Solution (KemEcal 142 | | |
| | PG available from Kemira Oyj) as the buffer instead of the citric acid solution, and a | | |
| | pH between 6.75-7.10 instead of 4.75-5.10 | | |
| | V = 1.44 wt % active (5:1 | W = 0.30 wt % active | Y = 0% |
| | Lactic Acid:Fructose) | (0.35 wt % formula) | |
| | | (Z = 20% solvent mixture) | |

*Z: Added as stated weight percent of the eutectic solvent mixture

TABLE 8
Turbidity and Appearance Data. Turbidity is measured in Nephelometric Turbidity
Units (NTU) using Hach 2100Q Portable Turbidimeter (Catalog # 2100Q01)

| | Turbidity | | | | |
|---|---|---|---|---|---|
| | (NTU) @ | Initial | 1 month stability | | |
| Formula | 1 week old | Appearance | 25° C. | 50° C. | 5° C. |
| L | 54.5 | Slightly hazy | Slightly hazy | Slightly hazy | Hazy |
| 21 | 42.2 | Slightly hazy | Slightly hazy | Slightly hazy | Slightly hazy |
| 22 | 44.6 | Slightly hazy | Slightly hazy | Slightly hazy | Slightly hazy |
| 23 | 40.5 | Clear | Clear | Clear | Slightly hazy |
| 24 | 46.4 | Slightly hazy | Slightly hazy | Slightly hazy | Slightly hazy |
| 25 | 36.1 | Clear | Clear | Clear | Clear |
| 26 | 33.9 | Clear | Clear | Clear | Clear |
| 27 | 30.0 | Clear | Clear | Clear | Slightly hazy |
| M | 41.7 | Hazy | Hazy | Hazy | Opaque |
| 28 | 43.2 | Slightly hazy | Slightly hazy | Slightly hazy | Slightly hazy |
| N | 362 | Cloudy with | NA | NA | NA |
| | | precipitate | | | |
| O | 28.8 | Clear | Clear | Clear | Slightly hazy |
| 29 | 30.0 | Clear | Clear | Clear | Clear |
| 30 | 31.0 | Clear | Clear | Clear | Slightly hazy |

The turbidity, stability, and initial appearance data support that formulations with the eutectic solvent and unmodified cyclodextrin (formulas 21-30) have a homogeneous initial appearance without opacity or precipitation as compared to Formula N (no eutectic solvent with unmodified cyclodextrin) which exhibited stability failure by forming a precipitate and having high turbidity.

In examples, the compositions in accordance with the present disclosure may have a turbidity at 1 week of from 10 to 150 NTU, from 10 to 100 NTU, from 10 to 90 NTU, from 10 to 80 NTU, from 30 to 130 NTU, from 30 to 100 NTU, from 30 to 90 NTU, from 30 to 80 NTU, from 50 to 130 NTU, from 50 to 100 NTU, from 50 to 90 NTU, from 50 to 80 NTU, or any values within the foregoing ranges or any ranges created thereby.

### Example 5: Method of Making a Composition in Accordance with the Present Disclosure

TABLE 9
Base formula 3

| | | Raw Material | Active | Formula |
|---|---|---|---|---|
| Material | Function | Activity | wt % | wt % |
| Perfume | Fragrance | 100.00% | 1.00% | 1.00% |
| Hydrogenated castor oil | Nonionic | 90.00% | 2.50% | 2.78% |
| (HCO) - Emulan ELH-60 | Surfactant | | | |
| available from BASF | | | | |
| Dowanol Eph6 glycol ether - | Solvent | 100% | 1.00% | 1.00% |
| available from Dow Chemical | | | | |
| Deionized Water | Solvent | 100.00% | balance | balance |
| Ethanol - available from | Solvent | 93.00% | 2.00% | 2.15% |
| Grain Processing Corporation | | | | |
| Lupasol HF- Available from | Malodor | 56.00% | 0.065% | 0.116% |
| BASF Ag | Reducer | | | |
| Silwet L-7600 - Available | Surfactant | 100.00% | (a) 0.05% | (a) 0.05% |
| from Momentive Performance | | | (b) 0.10% | (b) 0.10% |
| Materials | | | | |
| Uniquat 2250 - Available | Surfactant | 50.00% | 0.060% | 0.12% |
| from Arxada | | | | |
| Maleic Acid - Available from | Buffer | 30.00% | 0.06- | (a) 0.20% |
| Cymer LLC | | | 0.07% | (b) 0.23% |
| 1,2-benzisothiazolin-3-one | Preservative | 19.00% | 0.0044% | 0.023% |
| (BIT) - Koralone B-119 | | | | |
| available from LANXESS | | | | |
| Citric Acid Solution - | Buffer | 50.00% | 0.03% | 0.06% |
| available from Univar | | | | |
| Solutions | | | | |
| Eutectic Solvent (not | Solvent | 100.00% | V % | V % |
| including BCD) | | | | |
| Unmodified Beta Cyclodextrin | Malodor | 86.00% | W % | W % |
| (BCD) - Cavamax W7 | Reducer | | | |
| available from Wacker | | | | |
| Hydroxypropyl Beta | Malodor | 40.00% | Y % | Y % |
| Cyclodextrin (BCD) - Cavasol | Reducer | | | |
| W7 HP TL available from | | | | |
| Wacker | | | | |
| Sodium hydroxide - Formosa | pH adjustment | 5.00% | As needed | As needed |
| Plastics Corporation | | | for pH | for pH |

Table 9 shows the compositions for Base formula 3. There are two alternative base formulas, 3 (a) and 3 (b), which have differences in the levels of maleic acid and Silwet L-7600.
7. Perfume Premix. Weigh the specified quantity of perfume components, hydrogenated castor oil and half (0.50%) of the Dowanol Eph6 glycol ether into a suitably sized beaker. Add a stir bar to the mixture and place on a stir plate. Stir mixture at moderate speed for at least 15 minutes, or until ingredients are mixed thoroughly. Cover beaker and continue stirring mixture at room temperature (20-25 C) until it is used in step 11 below. Premix should be used within 4 hours after making.
8. In a separate glass beaker, add the specified amount of deionized water.
9. Use an IKA overhead mixer fitted with a pitched blade impeller (of suitable size to thoroughly blend the mixture) to begin stirring water at a speed which is adequate to form a mixing vortex in the center of the liquid mixture, but does not cause aeration of the liquid.
10. Using an analytical balance to weigh components, add the following components to the water in the following order. Continue overhead mixing and stir mixture for ˜5 minutes after addition of each component before adding the next component. Order of addition: ethanol, then Lupasol HF, then the second half (0.50%) of the Dowanol Eph6 glycol ether, then Silwet L-7600, then Uniquat 2250, then maleic acid, then Koralone B-119, and then citric acid.
11. Add the specified amount of the perfume premix prepared in step 7. Stir during addition and for ˜5 minutes after addition.
12. Add the specified amount of the following components to the mixture while continuing to stir mixture. Stir mixture for ˜5 minutes after addition of each component before adding the next component. Order of addition: Where one of (1) Eutectic Solvent mixture including Z active wt % unmodified betacyclodextrin is added; (2) hydroxypropyl betacyclodextrin without eutectic solvent is added; (3) unmodified betacyclodextrin powder without eutectic solvent is added; or (4) unmodified betacyclodextrin powder pre-dissolved in high pH (pH˜12) water is added.
13. Once all ingredients are added, continue stirring mixture for ˜5 minutes.
14 While continuing to stir mixture, use a calibrated pH probe to measure the pH of the mixture. If the pH is below 6.7, add 50% sodium hydroxide solution dropwise while continuing to stir and monitor the pH until the pH is 6.2-7.0.
15. Using a Hach 2100Q Portable Turbidimeter (Catalog #2100Q01) per manufacturer's instructions, measure the turbidity of the solution.

Packaging of formulations into jars or trigger spray bottles for stability assessment. Formulations with an initial appearance having a precipitate and/or severe phase separation can skip steps 16-19 and have stability marked as “NA”.
16. Obtain both a glass jar and a high density polyethylene bottle fitted with a trigger spray actuator. Add enough finished formula to the jar and to the bottle to fill it ½ to ¾ of the way full. Replace the actuator on the trigger spray bottle.
17. Store bottles containing finished formulas at the following constant temperature and/or temperature/humidity conditions: 5° C./60% relative humidity, 25° C./60% relative humidity, 50° C. and 3 cycles of freeze/thaw (24 hours at −18° C./24 hours at 15° C./60% relative humidity). Observe the visual appearance of the formula over time.

### Example 6: Stability Assessments for Eutectic Solvents Made Via the Process Above (Steps 1-5) in Example 1 and Added to the Base Formula 3 Via the Process Above in Example 5

TABLE 10
Formula compositions where the eutectic solvent ratios are molar ratios

| | V (wt % active | W (wt % active | Y (wt % active |
|---|---|---|---|
| Formula | Eutectic Solvent) | Unmodified BCD) | HPBCD) |
| P | Base Formula 3(a) with no eutectic solvent and with HPBCD | | |
| | V = 0% | W = 0% | Y = 0.63 wt % active |
| | | | (1.58 wt % formula) |
| 31 | Base Formula 3(a) with eutectic solvent and with unmodified BCD | | |
| | V = 3.47 wt % active (5:1 | W = 0.63 wt % active | Y = 0% |
| | Lactic Acid:Fructose) | (0.73 wt % formula) | |
| | | (Z = 15% solvent mixture*) | |
| 32 | Base Formula 3(a) with eutectic solvent and with unmodified BCD | | |
| | V = 3.47 wt % active (5:1 | W = 0.63 wt % active | Y = 0% |
| | Lactic Acid:Sorbitol) | (0.73 wt % formula) | |
| | | (Z = 15% solvent mixture) | |
| 33 | Base Formula 3(a) with no eutectic solvent and with unmodified BCD added | | |
| | separately to formula (not at Lactic acid:Fructose:BCD premix) | | |
| | V = 0% | W = 0.63 wt % active | Y = 0% |
| | | (0.73 wt % formula) | |
| | | (Z = 0% solvent mixture) | |
| 34 | Base Formula 3(a) with eutectic solvent and unmodified BCD powder added | | |
| | separately to formula (not at Lactic acid:Fructose:BCD premix) | | |
| | V = 2.50 wt % active (5:1 | W = 0.63 wt % active | Y = 0% |
| | Lactic Acid:Fructose) | (0.73 wt % formula) | |
| | | (Z = 0% solvent mixture) | |
| 35 | Base Formula 3(a) with eutectic solvent and unmodified BCD powder added | | |
| | separately to formula (not at Lactic acid:Fructose:BCD premix) | | |
| | V = 5.00 wt % active (5:1 | W = 0.63 wt % active | Y = 0% |
| | Lactic Acid:Fructose) | (0.73 wt % formula) | |
| | | (Z = 7% solvent mixture) | |
| 36 | Base Formula 3(a) with no eutectic solvent and with unmodified BCD powder pre- | | |
| | dissolved in high pH water added separately to formula (not at Lactic | | |
| | acid:Fructose:BCD premix) | | |
| | V = 0% | W = 0.63 wt % active | Y = 0% |
| | | (0.73 wt % formula) | |
| | | (Z = 15% solvent mixture) | |
| 37 | Base Formula 3(a) with eutectic solvent and unmodified BCD powder pre-dissolved | | |
| | in high pH water added separately to formula (not at Lactic acid:Fructose:BCD | | |
| | premix) | | |
| | V = 2.50 wt % active (5:1 | W = 0.63 wt % active | Y = 0% |
| | Lactic Acid:Fructose) | (0.73 wt % formula) | |
| | | (Z = 15% solvent mixture) | |
| 38 | Base Formula 3(a) with eutectic solvent and unmodified BCD powder pre-dissolved | | |
| | in high pH water added separately to formula (not at Lactic acid:Fructose:BCD | | |
| | premix) | | |
| | V = 5.00 wt % active (5:1 | W = 0.63 wt % active | Y = 0% |
| | Lactic Acid:Fructose) | (0.73 wt % formula) | |
| | | (Z = 15% solvent mixture) | |
| Q | Base Formula 3(b) with no eutectic solvent and with HPBCD | | |
| | V = 0% | W = 0% | Y = 0.90 wt % active |
| | | | (2.25 wt % formula) |
| 39 | Base Formula 3(b) with eutectic solvent and with unmodified BCD | | |
| | V = 4.95 wt % active (5:1 | W = 0.90 wt % active | Y = 0% |
| | Lactic Acid:Fructose) | (1.05 wt % formula) | |
| | | (Z = 15% solvent mixture*) | |
| 40 | Base Formula 3(b) with eutectic solvent and with unmodified BCD | | |
| | V = 3.45 wt % active (5:1 | W = 0.90 wt % active | Y = 0% |
| | Lactic Acid:Fructose) | (1.05 wt % formula) | |
| | | (Z = 20% solvent mixture*) | |
| 41 | Base Formula 3(b) with eutectic solvent and with unmodified BCD | | |
| | V = 4.95 wt % active (5:1 | W = 0.90 wt % active | Y = 0% |
| | Lactic Acid:Sorbitol) | (1.05 wt % formula) | |
| | | (Z = 15% solvent mixture*) | |

*Z: Added as stated weight percent of the eutectic solvent mixture

TABLE 11
Turbidity and Appearance Data. Turbidity is measured
in Nephelometric Turbidity Units (NTU) using Hach
2100Q Portable Turbidimeter (Catalog # 2100Q01)

| | Turbidity | | | | |
|---|---|---|---|---|---|
| | (NTU) @ | | | | |
| | 18 mos. | Initial | 1 month stability | | |
| Formula | old | Appearance | 25° C. | 50° C. | 5° C. |
| P | 7.5 | Clear | Clear | Clear | Clear |
| 31 | 8.1 | Clear | Clear | Clear | Slightly |
| | | | | | hazy |
| 32 | 8.4 | Clear | Clear | Clear | Clear |
| 33 | Opaque* | Clear with heavy | NA | NA | NA |
| | | precipitate | | | |
| 34 | Opaque* | Clear with heavy | NA | NA | NA |
| | | precipitate | | | |
| 35 | Opaque* | Clear with heavy | NA | NA | NA |
| | | precipitate | | | |
| 36 | 612 | Clear with heavy | NA | NA | NA |
| | | precipitate | | | |
| 37 | 129 | Hazy | NA | Clear with | NA |
| | | | | heavy | |
| | | | | precipitate | |
| | | | | @ 24 hrs | |
| 38 | 74.6 | Hazy | NA | Clear with | NA |
| | | | | heavy | |
| | | | | precipitate | |
| | | | | @24 hrs | |
| Q | 8.0 | Clear | Clear | Clear | Slightly |
| | | | | | hazy |
| 39 | 7.6 | Clear | Clear | Clear | Slightly |
| | | | | | hazy |
| 40 | 7.7 | Clear | Clear | Clear | Slightly |
| | | | | | hazy |
| 41 | 9.7 | Clear | Clear | Clear | Clear |

*Too high to measure

The turbidity, stability, and initial appearance data support that formulations with the eutectic solvent plus unmodified cyclodextrin made in-situ (formulas 31-32 and 39-41) have a homogeneous initial appearance without opacity or precipitation as compared to Formulas 33-38 (unmodified cyclodextrin and either no eutectic solvent or eutectic solvent added separately) which exhibited stability failure by forming a precipitate and having high turbidity.

In examples, the compositions in accordance with the present disclosure may have a turbidity at 1 week of from 10 to 150 NTU, from 10 to 100 NTU, from 10 to 90 NTU, from 10 to 80 NTU, from 30 to 130 NTU, from 30 to 100 NTU, from 30 to 90 NTU, from 30 to 80 NTU, from 50 to 130 NTU, from 50 to 100 NTU, from 50 to 90 NTU, from 50 to 80 NTU, or any values within the foregoing ranges or any ranges created thereby.

The presently described subject matter may include one or more aspects, which should not be regarded as limiting on the teaching of the present disclosure. A first aspect may include water; perfume; and a eutectic solvent comprising: a hydrogen bond acceptor comprising a monosaccharide, a disaccharide, or a sugar alcohol.

A second aspect may include a method of making a composition comprising: combining a hydrogen bond acceptor and a hydrogen bond donor to form a eutectic solvent, wherein the hydrogen bond acceptor comprises a monosaccharide, a disaccharide, or a sugar alcohol; adding the eutectic solvent to a perfume, forming a premix composition; and adding the premix composition to water to form the composition.

A third aspect may include a composition comprising: water; perfume; a eutectic solvent; and unmodified beta cyclodextrin.

A fourth aspect may include a method of making a composition comprising: combining a hydrogen bond acceptor, a hydrogen bond donor, and unmodified beta cyclodextrin to form a eutectic solvent, wherein the hydrogen bond acceptor comprises a monosaccharide, a disaccharide, or a sugar alcohol; and adding the eutectic solvent to water and a perfume, forming the composition.

Another aspect may include any of the previous aspects, wherein the eutectic solvent further comprises a hydrogen bond donor.

Another aspect may include any of the previous aspects, wherein the hydrogen bond donor comprises carboxylic acid.

Another aspect may include any of the previous aspects, wherein the hydrogen bond donor is selected from lactic acid, citric acid, and combinations thereof.

Another aspect may include any of the previous aspects, wherein the hydrogen bond donor to hydrogen bond acceptor are present at a molar ratio of from about 10:1 to about 1:1.

Another aspect may include any of the previous aspects, wherein the hydrogen bond donor to hydrogen bond acceptor are present at a molar ratio of from about 6:1 to about 4:1.

Another aspect may include any of the previous aspects, wherein the monosaccharaide, disaccharide, or sugar alcohol is selected from fructose, glucose, sucrose, xylose, sorbitol, mannose, galactose, xylitol, or combinations thereof.

Another aspect may include any of the previous aspects, wherein the composition comprises from 0.1 to 5 wt % unmodified beta-cyclodextrin.

Another aspect may include any of the previous aspects, wherein the composition further comprises from 0.1 to 5 wt % surfactant.

Another aspect may include any of the previous aspects, wherein the surfactant is selected from anionic surfactants, nonionic surfactants, cationic surfactants, amphoteric surfactants, zwitterionic surfactants, or combinations thereof.

Another aspect may include any of the previous aspects, wherein the composition comprises from 0.1 to 5 wt % perfume.

Another aspect may include any of the previous aspects, wherein the composition comprises from 70 to 99 wt % water.

Another aspect may include any of the previous aspects, wherein the composition comprises from 90 to 99 wt % water.

Another aspect may include any of the previous aspects, wherein the composition is aerosolized.

Another aspect may include any of the previous aspects, wherein the composition is contained in a spray container.

As used in this specification and the claims that follow, the articles “a”, “an”, and “the” include singular and plural references unless the context clearly dictates otherwise. As such, the terms “a” or “an”, “one or more” and “at least one” can be used interchangeably herein. Thus, for example, “a component” may include one or more components unless the reference is specifically indicated as being singular.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any examples disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular examples of the present composition have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A composition comprising:
water;
a eutectic solvent; and
low solubility cyclodextrin.

2. The composition of claim 1, wherein the eutectic solvent comprises a hydrogen bond donor and a hydrogen bond acceptor comprising a monosaccharide, a disaccharide, or a sugar alcohol.

3. The composition of claim 2, wherein the monosaccharide, disaccharide, or sugar alcohol is selected from fructose, glucose, sucrose, xylose, sorbitol, mannose, galactose, xylitol, or combinations thereof.

4. The composition of claim 2, wherein the hydrogen bond donor is selected from lactic acid, citric acid, salicylic acid, glycolic acid, 5 octanoyl salicylic acid, levulinic acid, hydroxyoctanoic acid, hydroxycaprylic acid, lanolin fatty acids, or combinations thereof.

5. The composition of claim 1, comprising from 0.1 to 5%, by weight of the composition, of the low solubility cyclodextrin, wherein the low solubility cyclodextrin comprises unmodified beta cyclodextrin.

6. The composition of claim 2, wherein the hydrogen bond donor and hydrogen bond acceptor are present at a molar ratio of from about 10:1 to about 2:1.

7. The composition of claim 2, wherein the hydrogen bond donor and hydrogen bond acceptor are present at a molar ratio of from about 6:1 to about 4:1.

8. The composition of claim 1, wherein the composition further comprises from 0.1 to 5%, by weight of the composition, of surfactant.

9. The composition of claim 8, wherein the surfactant is selected from anionic surfactants, nonionic surfactants, cationic surfactants, amphoteric surfactants, zwitterionic surfactants, or combinations thereof.

10. The composition of claim 1, wherein the composition further comprises perfume.

11. The composition of any claim 1, wherein the composition comprises from 70 to 99%, by weight of the composition, of the water.

12. The composition of any claim 1, wherein the composition comprises from 90 to 99%, by weight of the composition, of the water.

13. A method of making a composition comprising:
combining a hydrogen bond acceptor, a hydrogen bond donor, and low solubility cyclodextrin to form a eutectic solvent, wherein the hydrogen bond acceptor comprises a monosaccharide, a disaccharide, or a sugar alcohol; and
adding the eutectic solvent to water, forming the composition.

14. The method of claim 13, wherein the hydrogen bond donor is selected from lactic acid, citric acid, salicylic acid, glycolic acid, 5 octanoyl salicylic acid, levulinic acid, hydroxyoctanoic acid, hydroxycaprylic acid, lanolin fatty acids, or combinations thereof.

15. The method of claim 13, wherein the composition comprises from 0.1 to 5%, by weight of the composition, of the low solubility cyclodextrin, wherein the low solubility cyclodextrin comprises unmodified beta cyclodextrin.

16. The method of claim 13, wherein the hydrogen bond donor and hydrogen bond acceptor are present at a molar ratio of from about 8:1 to about 2:1.

17. The method of claim 13, wherein the hydrogen bond donor and hydrogen bond acceptor are present at a molar ratio of from about 6:1 to about 4:1.

18. The method of claim 13, wherein the monosaccharaide, disaccharide, or sugar alcohol is selected from fructose, glucose, sucrose, xylose, sorbitol, mannose, galactose, xylitol, or combinations thereof.

19. The method of claim 13, wherein the method comprises adding the eutectic solvent to the water and a perfume, forming the composition.

20. The method of claim 13, wherein the composition comprises from 70 to 99%, by weight of the composition, of the water.
